(19) United States
(12) Patent Application Publication
(10) Pub. No.: US 2013/0116656 A1
(43) Pub. Date: May 9, 2013

(54) Nose Bleeding Stopper.

(71) Applicant: Yong Song, Fremont, CA (US)

(72) Inventor: Yong Song, Fremont, CA (US)

(21) Appl. No.: 13/291,110

(22) Filed: Nov. 8, 2011

(51) Int. Cl. A61M 31/00; A61M 29/00

(52) U.S. Cl. 604/514; 606/199; 604/285

(57) ABSTRACT

The present invention discloses a nose plug device comprising a compressible material to exert a pressure on bleeding areas in inner wall of nostril. The present nose plug device can further comprise a bio-lubricant coating to help in device removal.

### BACKGROUND OF THE INVENTION

The present invention relates to a plug, more particularly to a nose plug device allowing users to plug nostril to stop nostril bleeding.

### SUMMARY OF THE DESCRIPTION

In some embodiments, the present invention discloses a nose plug, nasal depository, or the like to stop nostril bleeding. The nose plug or nasal depository comprises a compressible material to apply a force on the bleeding area inside the nostril, together with a surface coating of a bio-lubricant to ease of removal.

In some embodiments, the nose plug further comprises a venting structure for breathing or for draining fluids within the nostril, such as blood or nasal discharge. The nose plug material can comprise an absorbent material for absorbing fluids within the nostril, such as blood or nasal discharge.

In some embodiments, the present invention discloses a single nose plug, or a pair of nose plugs coupled to each other. In some embodiments, the present invention discloses methods to apply the nose plug.

### BRIEF DESCRIPTION OF THE DRAWINGS

FIGS. 1A-1B illustrate an exemplary nose plug device according to some embodiments of the present invention.

FIGS. 2A-3B illustrate another exemplary nose plug device according to some embodiments of the present invention.

FIGS. 3A-3B illustrate another exemplary nose plug device according to some embodiments of the present invention.

FIGS. 4A-4C illustrate an exemplary nose plug having cone shape according to some embodiments of the present invention.

FIGS. 5A-5B illustrate exemplary views of the nose plug in nostril.

FIGS. 6A-6D illustrate an exemplary delivery service for a nose plug according to some embodiments of the present invention.

FIGS. 7A-7B illustrate exemplary flowcharts for using a nose plug according to some embodiments of the present invention.

### DETAILED DESCRIPTION OF THE PREFERRED EMBODIMENTS

The present invention relates to a plug, more particularly to a nose plug device allowing users to plug nostril to stop nostril bleeding, such as a nasal depository for absorbing blood and the like from a person nose.

In some embodiments, the nose plug device comprises a compressible material that expands upon releasing to the nostril, which can produce a compression pressure on bleeding area in inner wall of nostril. For example, the device can have a porous structure lake foam which allows the device to be physically compressed. The compressed foam can be recovered to an uncompressed natural size after releasing. This foam recovery force or strength serves as constant compression pressure on bleeding area inside nostril, achieving effect of stopping bleeding. The porous structure of the device can have a function of absorbing blood.

In some embodiments, the device material can also be pre-dehydrated in a pre-compressed form. The device can immediately swell once contacting with blood or moisture. In some embodiments, the device can also be formed in a firm (solid) version instead of compressible foam. Both soft and firm plugs come with different sizes.

In some embodiments, the present invention discloses a single nose plug, or a pair of nose plugs coupled to each other. A single nose plug device may be employed in the circumstances or situations where there would be need for only a single nose plug, such as blood originating through only one nasal passageway. Alternatively, the nose plug device can comprise an integrally shaped nose plug which comprises a connecting piece and a pair of nose plugs on opposing sides. A coupling device might be provided to connect the two nose plugs. The coupling device might also be used to prevent the nose plug from being deeply engaged into the nose.

The device can be made of polymer materials such as polyvinyl alcohol (PVA), polyvinyl chloride (PVC), polyurethane (PU), polyethylene (PE), silicone rubber and hydro gel et al. It can be also made of natural fabrics such as cotton, linen et al.

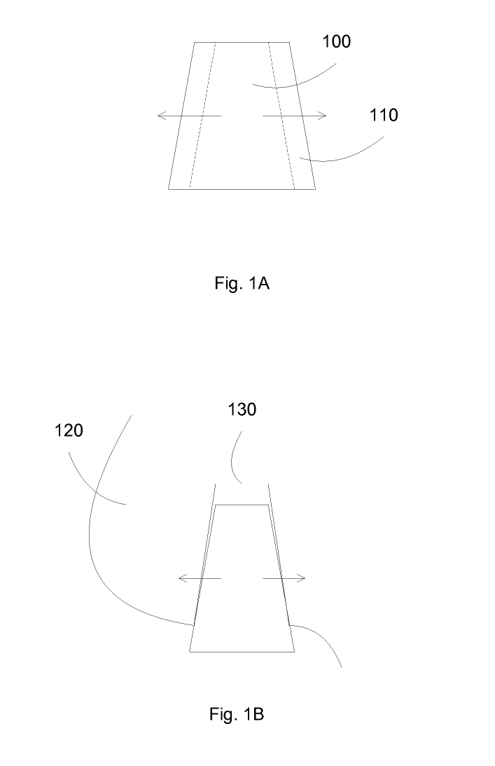

FIGS. 1A-1B illustrate an exemplary nose plug device according to some embodiments of the present invention. A nose plug device 100 comprises compressible material that can be expanded 110. Upon inserted in to nostril passageway 130 of a person's nose 120, the nose plug can exert pressure on the passageway, helping in stop of the bleeding.

The nose plug is formed in such as to be easily inserted into a nasal passageway. In some embodiments, in order to remove the device smoothly without causing sealed, coagulated bleeding site to bleed again, the outer surface of the device can be coated with bio-lubricants such as Vaseline, silicone gel or Teflon. Further, the device surface can be coated with coagulants, such as CELOX and thrombin.

CELOX is a new granular hemostatic agent that can provide fast bleeding control by forming a gel-like mass over the bleeding site to protect the wound. It can be easy to clean. It is versatile, and can be used in low temperatures, high pressure, or high volume bleeding from major arteries.

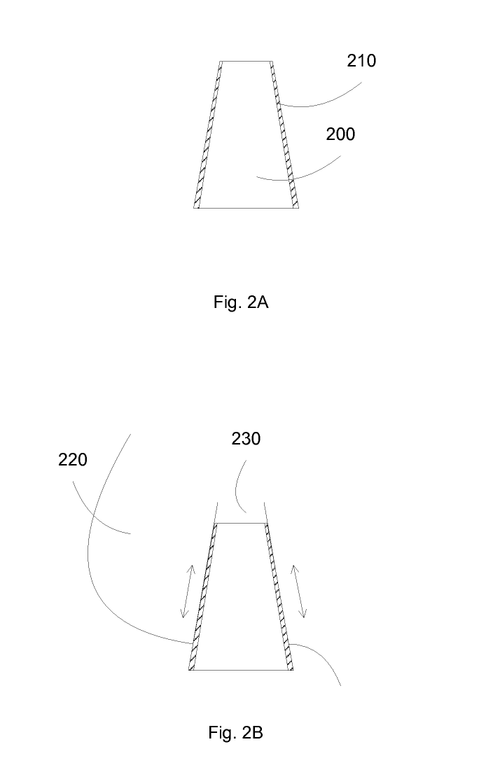

FIGS. 2A-2B illustrate an exemplary nose plug device according to some embodiments of the present invention. A nose plug device 200 comprises a bio-lubricant coating 210. Upon inserted in to nostril passageway 230 of a person's nose 220, the nose plug can be easily removed without re-opening the bleeding sites.

In some embodiments, one or more hollow venting structures are extended through the nose plug which functions to allow the user of nasal depository to easily breathe when the nostrils contain the nasal depository. In some embodiments, the slightly oversized firm plug is inserted into nostril. The stretched wall of nostril creates compression pressure on bleeding area. Both soft and solid versions may have a venting structure for allowing users to breathe in or out. This structure can be a tubular structure in the center of the device. The residual blood and nasal discharge can be drained out through the hole. Venting tube material can be metal or polymer. The tubular structure can be designed like a stent that is expendable in diameter. The nose plug can have an absorbent member for absorbing blood when in contact with nasal blood/fluid. The tubular structure can be configured to be unobstructed by the absorbent member to allow the user to readily breathe.

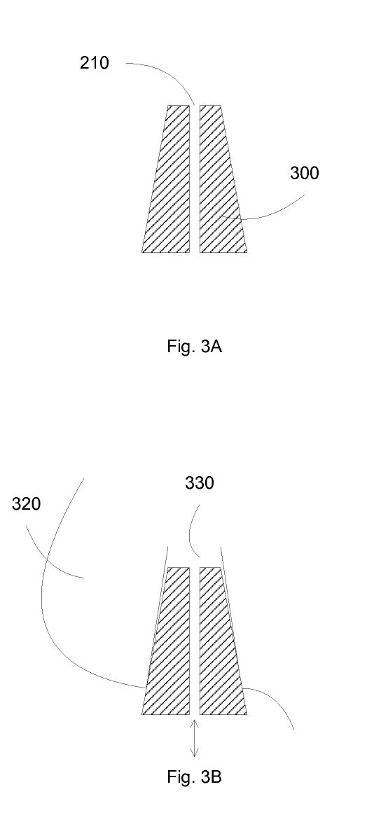

FIGS. 3A-3B illustrate an exemplary nose plug device according to some embodiments of the present invention. A nose plug device 300 comprises a venting structure 110. Upon inserted in to nostril passageway 330 of a person's nose 320, the venting structure can allow drainage of the nasal fluid, such as blood, or to help the person breathing.

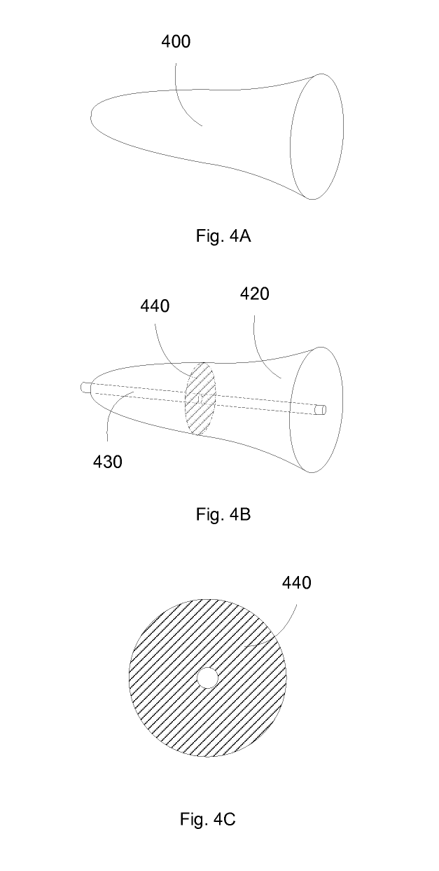

FIGS. 4A-4C illustrate an exemplary nose plug having cone shape according to some embodiments of the present invention. In some embodiments, the plug is shaped to fit natural anatomy of nostril. FIG. 4A shows an exemplary nose plug 400 without any venting hole. FIG. 4B shows an exemplary nose plug 420 with a venting hole 430. FIG. 4C shows a cross section 440 of the plug.

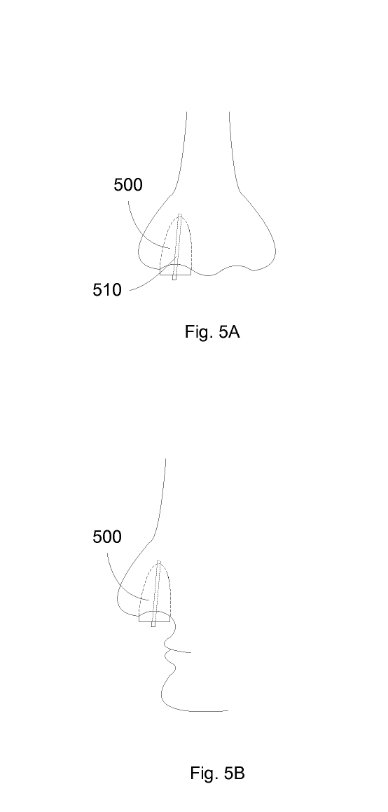

FIGS. 5A-5B illustrate exemplary views of the nose plug in nostril. FIG. 5A shows an exemplary nose plug 500 having venting hole 510 in an anterior-posterior view of the plug in nostril. FIG. 5B shows a lateral view of the plug 500 in nostril.

In some embodiments, the present invention discloses a delivery service to deliver the nose plug to a nostril. The nose plug can be compressed and preloaded to a delivery device. After positioning the delivery at the nostril, the nose plug can be pushed into the nostril, or the delivery device can be retrieved. In some embodiments, the delivery device is first inserted into the nostril, either partially or completely.

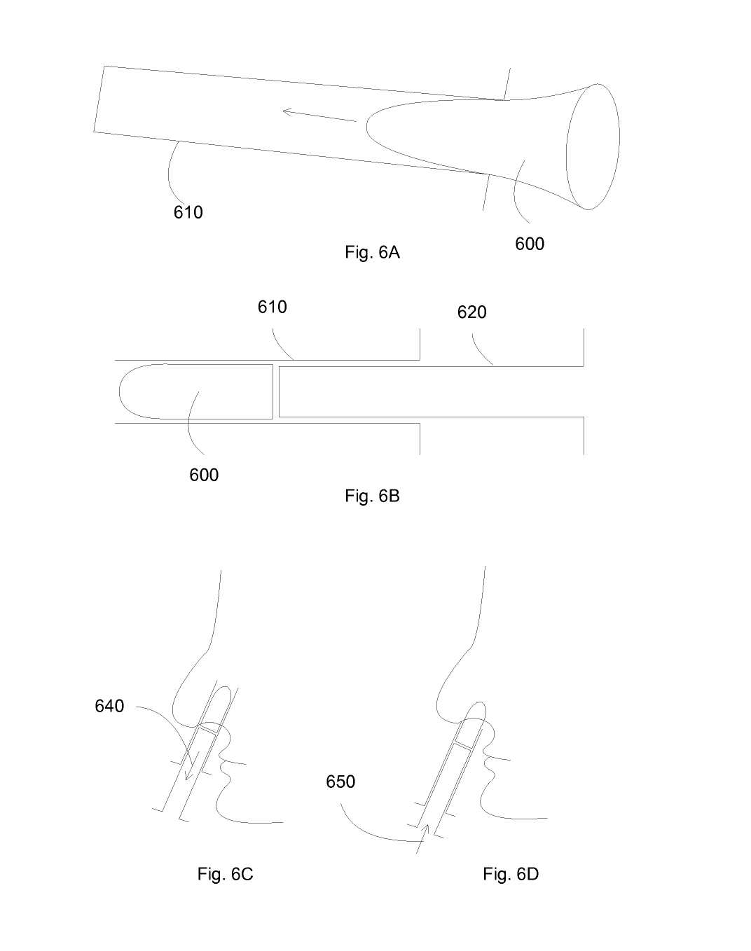

FIGS. 6A-6D illustrate an exemplary delivery service for a nose plug according to some embodiments of the present invention. In FIG. 6A, a nose plug 600 is preloaded in a tubular delivery device 610. The nose plug 600 is preferably preloaded in compressed form, for example, the diameter of the tubular delivery device 610 is smaller than the corresponding diameter of the nose plug 600, thus the nose plug is compressed during the loading process.

In FIG. 6B, the nose plug 610 is preferably disposed at an end of the tubular delivery device 610. A handle 620 can be used to push the nose plug 600 along the interior of the tubular delivery device 610.

In FIG. 6C, the tubular delivery device is partially inserted into the nostril. The tubular delivery device then withdrawn 640, leaving the nose plug situated in the nostril. The handle 620 can be pushed against the nose plug to prevent the nose plug from being sliding outward together with the tubular delivery device. FIG. 6D shows an alternative process of inserting the nose plug into the nostril. The tubular delivery device, together with the preloaded nose plug, is positioned at the vicinity of the nostril. The compressed nose plug is pushed 650 out of the tubular delivery device, and into the nostril, for example, by the same handle 620.

The nose plug then can expanded, e.g., released from the compressed from, to fill the interior volume of the nostril. The nose plug is preferably larger than the nostril volume, thus exerting a compressive pressure on the interior wall of the nostril, helping stop the bleeding.

In some embodiments, the outside surface of the nose plug is coated with a bio-lubricant, which can help in the process of preloading to the delivery device. Further, the lubricant coating can also help in the nose plug removal process, for example, assisting in preventing being attached to the skin, and to eliminate or reducing friction in removing the nose plug. The lubricant coating can also eliminate or reducing the bonding of the coalescent blood to the nose plug, facilitating the removal process.

In some embodiments, the outside surface of the nose plug is coated with a coagulant, which can help in stopping the flow of blood in the nostril. In some embodiments, the nose plug comprises a venting hole.

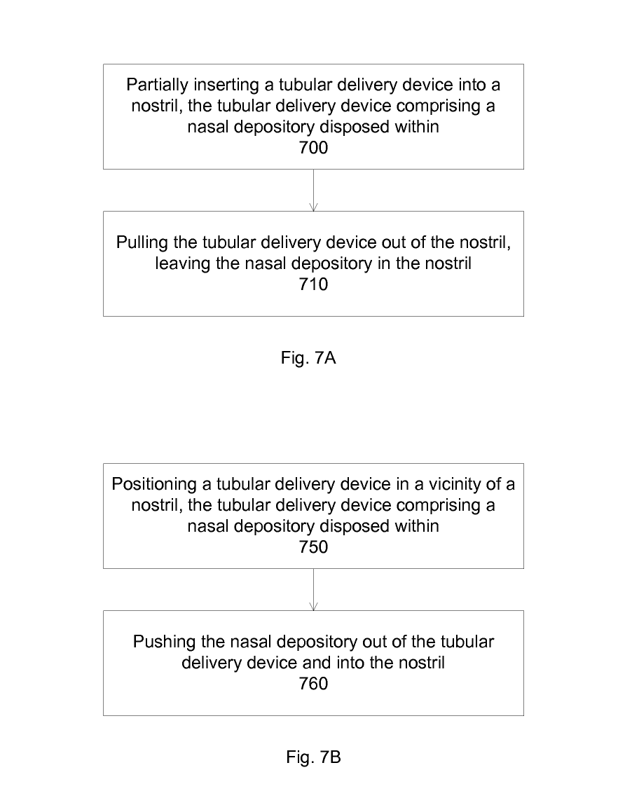

FIGS. 7A-7B illustrate exemplary flowcharts for using a nose plug according to some embodiments of the present invention. In FIG. 7A, operation 700 partially inserts a tubular delivery device into a nostril, the tubular delivery device comprising a nasal depository disposed within. In operation 710, the tubular delivery device is pulled out of the nostril, leaving the nasal depository in the nostril. In some embodiments, the method further comprises supporting the nasal depository to prevent the nasal depository from being pulling out together with the tubular delivery device. The method can further comprise preloading the nasal depository to the interior of the tubular delivery device. The method can further comprise compressing the nasal depository when preloading to the interior of the tubular delivery device. The method can further comprise applying a bio-lubricant coating on an outside surface portion of the nasal depository. The method can further comprise applying a coagulant coating on an outside surface portion of the nasal depository. In some embodiments, the nasal depository comprises a venting structure.

In FIG. 7A, operation 750 positions a tubular delivery device in a vicinity of a nostril, the tubular delivery device comprising a nasal depository disposed within. In operation 760, the nasal depository is pushed out of the tubular delivery device and into the nostril. In some embodiments, the method further comprises supporting the nasal depository to prevent the nasal depository from being pulling out together with the tubular delivery device. The method can further comprise preloading the nasal depository to the interior of the tubular delivery device. The method can further comprise compressing the nasal depository when preloading to the interior of the tubular delivery device. The method can further comprise applying a bio-lubricant coating on an outside surface portion of the nasal depository. The method can further comprise applying a coagulant coating on an outside surface portion of the nasal depository. In some embodiments, the nasal depository comprises a venting structure.

What is claimed is:

1. A nasal depository for stop nostril bleeding, comprising
a compressible material for exerting a pressure on the inside of a nostril;
a bio-lubricant coating on an outside portion of the compressible material.

2. A nasal depository as in claim 1, wherein the compressible material comprises a porous foam, an absorbent material, or a material in dehydrated form.

3. A nasal depository as in claim 1, wherein the compressible material comprises PVC, PU, PE, silicone rubber or hydro gel.

4. A nasal depository as in claim 1, wherein the compressible material comprises natural fabrics, cotton, or linen.

5. A nasal depository as in claim 1, further comprising
a venting structure within the compressible material.

6. A nasal depository as in claim 5, wherein the venting structure comprises a metal or polymer.

7. A nasal depository as in claim 1, further comprising
a coagulant coating on an outside portion of the compressible material.

8. A nasal delivery comprising
a tubular delivery device;
a nasal depository disposed within the tubular delivery device, the nasal depository being compressed;
a handle slidably within the tubular delivery device for pushing the nasal depository out of the tubular delivery device.

9. A nasal delivery as in claim 8, wherein the nasal depository comprises a bio-lubricant coating on an outside surface portion.

10. A nasal delivery as in claim 8, wherein the nasal depository comprises a coagulant coating on an outside surface portion.

11. A nasal delivery as in claim 8, wherein the nasal depository comprises a compressible material.

12. A nasal delivery as in claim 8, wherein the nasal depository comprises a venting structure.

13. A method to deliver a nasal depository comprising
partially inserting a tubular delivery device into a nostril, the tubular delivery device comprising a nasal depository disposed within;
pulling the tubular delivery device out of the nostril, leaving the nasal depository in the nostril.

14. A method as in claim 13, further comprising
supporting the nasal depository to prevent the nasal depository from being pulling out together with the tubular delivery device.

15. A method as in claim 13, further comprising
preloading the nasal depository to the interior of the tubular delivery device.

16. A method as in claim 13, further comprising
compressing the nasal depository when preloading to the interior of the tubular delivery device.

17. A method as in claim 13, further comprising
applying a bio-lubricant coating on an outside surface portion of the nasal depository.

18. A method as in claim 13, further comprising
applying a coagulant coating on an outside surface portion of the nasal depository.

19. A method as in claim 13, wherein the nasal depository comprises a venting structure.

20. A method to deliver a nasal depository comprising
positioning a tubular delivery device in a vicinity of a nostril, the tubular delivery device comprising a nasal depository disposed within;
pushing the nasal depository out of the tubular delivery device and into the nostril.
